(19) United States
(12) Patent Application Publication
(10) Pub. No.: US 2011/0295317 A1
(43) Pub. Date: Dec. 1, 2011

(54) Restraint system for a spinal decompression table

(76) Inventor: Richard E. Busch, III, Spencerville, IN (US)

(21) Appl. No.: 13/068,965

(22) Filed: May 25, 2011

Related U.S. Application Data
(60) Provisional application No. 61/347,923, filed on May 25, 2010.

(51) Int. Cl. A61F 5/00

(52) U.S. Cl. 606/241

(57) ABSTRACT

A spinal decompression table includes a table frame, a back support portion attached to the table frame and defining a longitudinal direction, at least one pair of arms coupled with and extending from the back support portion. When in a release position, the at least one pair of arms is generally coplanar with the back support portion. Each pair of arms has a pair of respective distal ends which are substantially aligned relative to the longitudinal direction. The respective distal ends of each of the at least one pair of arms are removably coupled together with a securing mechanism.

### CROSS REFERENCE TO RELATED APPLICATIONS

This is a non-provisional application based upon U.S. provisional patent application Ser. No. 61/347,923, entitled “SPINAL DECOMPRESSION RESTRAINT SYSTEM”, filed May 25, 2010, which is incorporated herein by reference.

### BACKGROUND OF THE INVENTION

1. Field of the Invention

The present invention relates to a spinal decompression system and, more particularly, to a restraint system for a spinal decompression table.

2. Description of the Related Art

Spinal decompression tables are non-surgical devices which leverage the natural gravity and weight of a patient's body to create negative intradiscal pressure, elongate the spine and alleviate disc compression in the back and neck. This provides at least temporary relief from pain and allows herniated and degenerative discs to heal without the necessity of invasive surgical procedures.

Existing spinal decompression tables are known to utilize complex harness systems to secure a patient to the spinal decompression table. Numerous problems have been associated with use of this type of restraint system. For example, the harness system is very cumbersome and uncomfortable. In addition, a substantial amount of time is expended to secure the patient in the harness itself and then to further secure the harness to the spinal decompression table. It is also difficult to achieve consistency of treatment for a patient from one visit to the next as there is no mechanism for measuring and applying a consistent, predefined amount of pressure or tension to the restraint system, thereby ensuring that the patient is firmly secured to the spinal decompression table and that the treatment administered is uniform, if so desired.

Another form of restraint system for existing spinal decompression tables includes the utilization of belts. Numerous problems have also been associated with this type of restraint system. Over time, due to the application of tension to these belts they become stretched and malformed, shortening the effective lifespan of the restraint and rendering the belts ineffective for their intended use. Additionally, these belts can be uncomfortable for a patient already suffering from back and/or neck pain.

What is needed in the art is a spinal decompression table which includes a restraint system that provides a sufficient level of comfort for a patient while ensuring that the patient is firmly secured to the spinal decompression table with a minimum expenditure of time.

### SUMMARY OF THE INVENTION

The present invention provides a spinal decompression table and a method of securing a patient to a spinal decompression table.

The invention in one form is directed to a spinal decompression table including a table frame and a back support portion attached to the table frame. The back support portion defines a longitudinal direction. At least one pair of arms is coupled with and extends from the back support portion and is generally coplanar with the back support portion when in a release position. In other words, although the arms may include some curvature, the arms are generally and substantially coplanar with the back support portion. Each of the at least one pair of arms has a pair of respective distal ends which are substantially aligned relative to the longitudinal direction. A securing mechanism is provided for removably coupling together the distal ends of each of the at least one pair of arms.

The invention in another form is directed to a spinal decompression table including a table frame and a back support portion attached to the frame, the back support portion defining a longitudinal direction. A pelvic portion including at least one pair of pelvic arms is coupled with and extends from the back support portion. The at least one pair of pelvic arms are generally coplanar with the back support portion when in a release position. Each of the at least one pair of pelvic arms has a pair or respective distal ends which are substantially aligned relative to the longitudinal direction. The spinal decompression table further includes a thoracic portion including at least one pair of thoracic arms coupled with and extending from the back support portion. The at least one pair of thoracic arms is also generally coplanar with the back support portion when in a release position. Each of the at least one pair of thoracic arms has a pair of respective distal ends which are substantially aligned relative to the longitudinal direction. A pelvic securing mechanism is provided for removably coupling the respective distal ends of each pair of pelvic arms and a thoracic securing mechanism is provided for removably coupling the respective distal ends of each pair of thoracic arms.

The present invention further provides a method of restraining a patient to a spinal decompression table which includes the step of positioning a patient on a back support portion which is attached to a table frame, the back support portion defining a longitudinal direction. At least one pair of arms coupled with and extending from the back support portion is then positioned around a body of the patient such that a pair of respective distal ends of the at least one pair of arms is substantially aligned relative to the longitudinal direction. The pair of respective distal ends of the at least one pair of arms are removably coupled with a securing mechanism and a predefined amount of tension is applied through a biasing mechanism to the securing mechanism in order to firmly secure the body of the patient to the spinal decompression table.

An advantage of the present invention is the spinal decompression table provides an efficient system for firmly securing a patient.

Another advantage is that the back support portion, the at least one pair of arms and the securing mechanism according to the present invention may be retrofitted for different types of table frames or spinal decompression tables.

Another advantage is that since the back support portion, arms and securing mechanism are physically attached to the spinal decompression table, a patient may be quickly secured and firmly attached to the spinal decompression table.

Yet another advantage is the present invention overcomes the problems known in the existing art relative to slack present in canvas or leather belt harness systems due to the rigid or semi-rigid pair(s) of arms which extend around the body of the patient to secure the patient to the spinal decompression table.

A further advantage of the present invention is that through the application of a predefined amount of tension to the securing mechanism via a biasing member, consistent pressure may be applied to a patient from one visit to the next, thereby ensuring that a patient is securely attached to the spinal decompression table.

### BRIEF DESCRIPTION OF THE DRAWINGS

The above-mentioned and other features and advantages of this invention, and the manner of attaining them, will become more apparent and the invention will be better understood by reference to the following description of embodiment of the invention taken in conjunction with the accompanying drawings, wherein:

FIG. 1 is a perspective view of a spinal decompression table according to the present invention in a release position;

FIG. 2 is a top view of a spinal decompression table according to the present invention in a release position

FIG. 3 is a perspective view of a spinal decompression table according to the present invention in a secure position;

FIG. 4 is a perspective view of a biasing member according to the present invention;

FIG. 5 is a sectional view of a spinal decompression table according to the present invention; and

FIG. 6 is a spinal decompression table according to the present invention.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

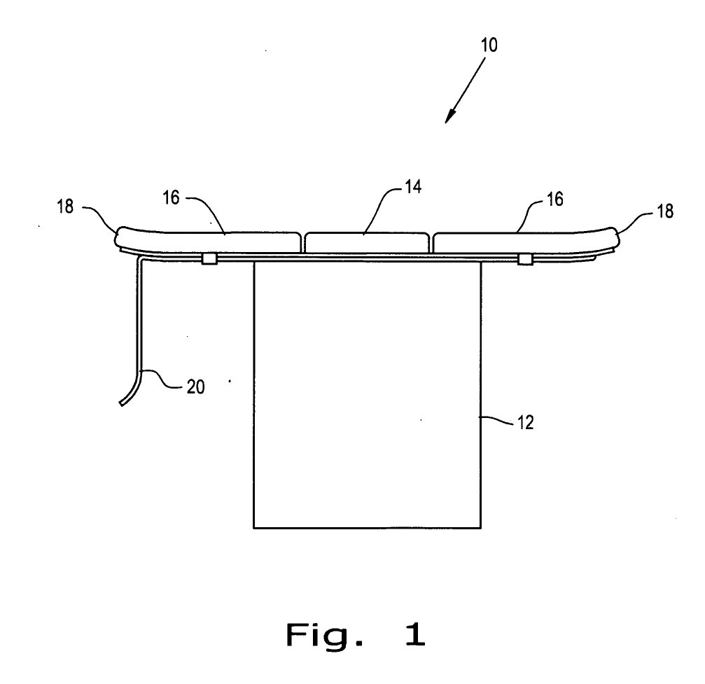

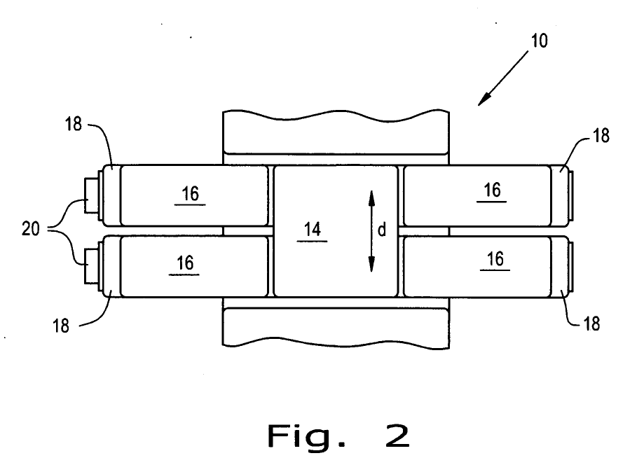

Referring now to the drawings, and more particularly to FIGS. 1 and 2, there is shown spinal decompression table 10 which generally includes table frame 12, back support portion 14 and at least one pair of arms 16 extending from back support portion 14. Back support portion 14 defines a longitudinal direction d, the at least one pair of arms 16 being substantially aligned relative to longitudinal direction d. Spinal decompression table 10 further includes a securing mechanism 20 for removably coupling together respective distal ends 18 of the at least one pair of arms 16.

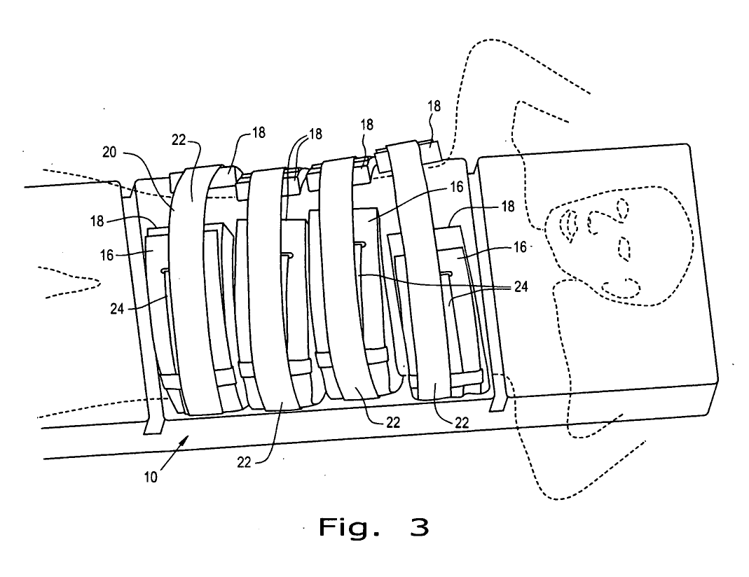

Referring now to FIG. 3, there is shown spinal decompression table 10 in a secure position. As illustrated, in a secure position, the at least one pair of arms 16 wrap around the body of a patient such that the respective distal ends 18 extend toward each other and are substantially aligned. The respective distal ends 18 are securely coupled together with securing mechanism 20. In one embodiment of the present invention, the securing mechanism 20 includes a strap 22 and a fastener 24. Fastener 24 is illustrated as being in the form of a hook and loop fastener, however, fastener 24 may, for example, be in the form of a button or buttons, a zipper, laces or a buckle or any known fastener.

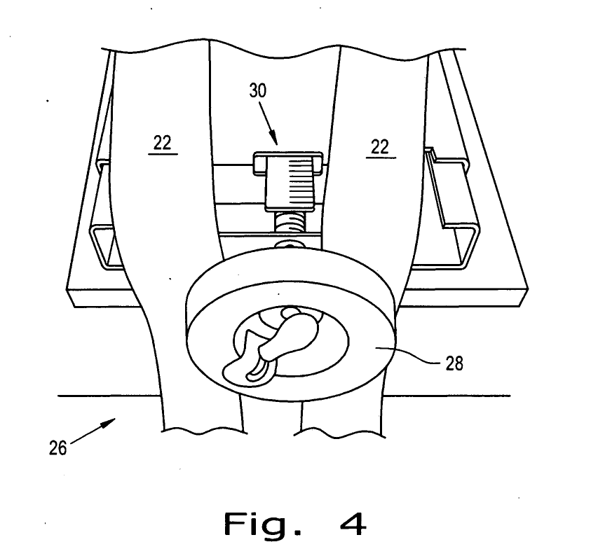

Referring now to FIG. 4, there is shown biasing member 26 which may further be utilized to apply tension to strap 22 in order to firmly secure a patient to spinal decompression table 10. Biasing member 26 is illustrated in FIG. 4 as roller 28 for placing tension on strap 22, but could be differently configured depending upon the application. Optionally, a gauge 30 may be provided for measuring the amount of tension applied to securing mechanism 20. Advantageously, the use of gauge 30 allows a predefined amount of tension to be applied to a particular patient from one use to the next, thereby providing for consistency of treatment and ensuring that the patient is firmly secured to the spinal decompression table 10.

In the embodiments of the present invention shown in each of FIGS. 1, 2, 3, 5 and 6, the at least one pair of arms 16 are pivotally connected to back support portion 14. In another embodiment of the present invention (not shown), the at least one pair of arms 16 are slidably movable relative to back support portion 14. Further, arms 16 generally include an arm base, a layer of memory foam and a covering positioned over the arm base and the memory foam. The arm base is, for example, a rigid plastic, steel or stainless steel. The arm base may further be semi-rigid such that sufficient flexibility is provided to wrap the arm around the body of a patient and, thus, firmly secure the patient to spinal decompression table 10.

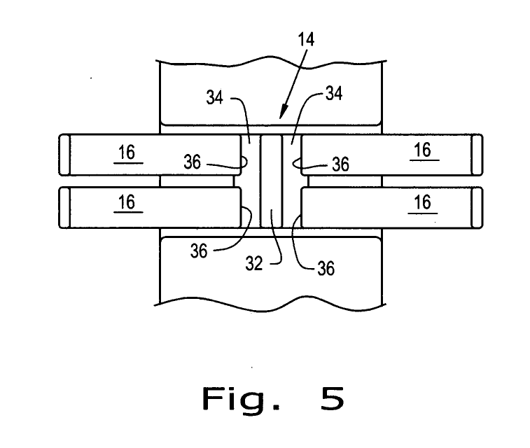

Back support portion 14 may be permanently affixed or removably coupled with table frame 12. Referring now to FIG. 5, there is shown back support portion 14 coupled with at least one pair of arms 16 so as to allow sufficient mobility for arms 16 to wrap around the body of a patient when in a secure position. Back support portion 14 may be the form of a single piece or multiple pieces. For example, back support portion 14 may include a central back support portion 32 and a pair of webbing portions 34 which are coupled with central back support portion 32 on each side and aligned in longitudinal direction d. Webbing portions 34 are further coupled with respective proximal ends 36 of the at least one pair of arms 16 such that webbing portions 34 extend between central back support portion 32 and arms 16.

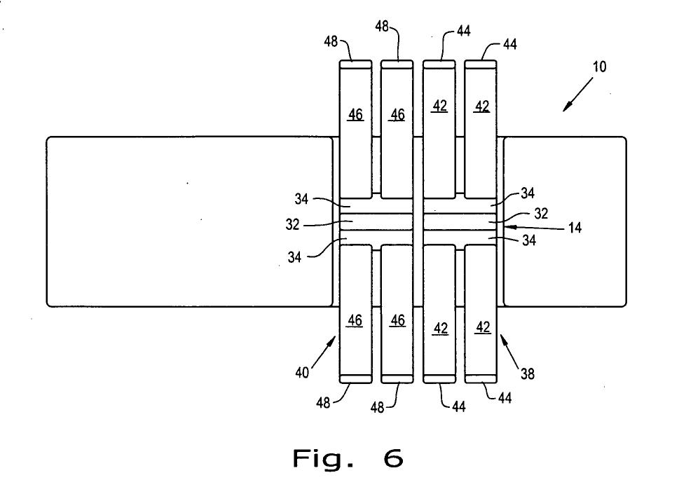

Referring now to FIG. 6, there is shown an embodiment of the spinal decompression table 10 according to the present invention including table frame 12, back support portion 14, thoracic portion 38 and pelvic portion 40. Thoracic portion 38 includes at least one pair of thoracic arms 42 moveably coupled with and extending from back support portion 14. Each of the at least one pair of thoracic arms 42 has a pair of respective distal ends 44 which are substantially aligned relative to longitudinal direction d. Further, pelvic portion 40 includes at least one pair of pelvic arms 46 having a pair of respective distal ends 48 which are substantially aligned relative to longitudinal direction d and which are moveably coupled with and extending from back support portion 14.

Another embodiment of the present invention further provides that back support portion 14 may be divided into a first portion coupled with thoracic portion 38 and a second portion coupled with pelvic portion 40, as illustrated in FIG. 6. Each of the first portion and the second portion of back support portion 14 may further include central back support portion 32 and webbing portions 34 to provide for flexibility of movement between the thoracic arms 42 and pelvic arms 46 respectively and the back support portion 32.

In use, a patient is positioned on back support portion 14. Back support portion 14 defines longitudinal direction d and is attached to table frame 12. At least one pair of arms 16, coupled with and extending from back support portion 14, is positioned around the body of the patient such that the respective distal ends 18 of the at least one pair of arms 16 are substantially aligned relative to longitudinal direction d. Distal ends 18 are then removably coupled together with securing mechanism 20. A predefined amount of tension is applied to securing mechanism 20 through biasing member 26 to firmly secure the body of the patient to spinal decompression table 10.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A spinal decompression table, comprising:
a table frame;
a back support portion defining a longitudinal direction, said back support portion configured to be attached to said frame;
at least one pair of arms coupled with and extending from said back support portion, said at least one pair of arms when in a release position being generally coplanar with said back support portion and each said pair of arms having a pair of respective distal ends which are substantially aligned relative to said longitudinal direction; and
a securing mechanism for removably coupling together said distal ends of each of said at least one pair of arms.

2. The spinal decompression table according to claim 1, wherein said at least one pair of arms are one of slidably movable and pivotally connected to said back support portion.

3. The spinal decompression table according to claim 1, wherein in a secure position, said pair of arms is configured to wrap around the body of a patient such that said respective distal ends of said at least one pair of arms extend towards each other and are substantially aligned.

4. The spinal decompression table according to claim 1, wherein said back support portion is one of permanently affixed and removably coupled with said table frame.

5. The spinal decompression table according to claim 1, wherein said securing mechanism includes a strap and a fastener.

6. The spinal decompression table according to claim 5, wherein said fastener is one of a hook and loop fastener, at least one button, a zipper, laces and a buckle.

7. The spinal decompression table according to claim 5, further comprising a biasing member through which tension is applied to said securing mechanism.

8. The spinal decompression table according to claim 7, wherein said biasing member includes said strap and a roller for placing tension on said strap.

9. The spinal decompression table according to claim 7, further comprising a gauge for measuring an amount of said tension applied to said securing mechanism.

10. The spinal decompression table according to claim 1, where each of said at least one pair of arms further comprises an arm base, a layer of memory foam and a covering positioned over said arm base and said memory foam.

11. The spinal decompression table according to claim 1, wherein said arm base is one of a rigid plastic, steel and stainless steel.

12. The spinal decompression table according to claim 1, wherein said back support portion includes a central back support portion and a webbing portion on each side of said central back support portion, said webbing portion being configured to provide flexibility of movement of said at least one pair of arms relative to said central portion.

13. A spinal decompression table, comprising:
a table frame;
a back support portion defining a longitudinal direction and coupled with said table frame;
a pelvic portion including at least one pair of pelvic arms coupled with and extending from said back support portion, said at least one pair of pelvic arms being generally coplanar with said back support portion when in a release position, each of said pair of pelvic arms having a pair of respective distal ends which are substantially aligned relative to said longitudinal direction;
a thoracic portion including at least one pair of thoracic arms coupled with and extending from said back support portion, said at least one pair of thoracic arms being generally coplanar with said back support portion when in a release position, each of said pair of thoracic arms having a pair of respective distal ends which are substantially aligned relative to said longitudinal direction;
a pelvic securing mechanism for removably coupling together said distal ends of each of said pair of pelvic arms; and
a thoracic securing mechanism for removably coupling together said distal ends of each of said pair of thoracic arms.

14. The spinal decompression table according to claim 13, wherein said back support portion includes a central portion and a webbing portion on each side of said central portion relative to said longitudinal direction, said webbing portion on each side of said central portion coupling said central portion with an arm of said at least one pair of thoracic arms and providing flexibility of movement between said at least one pair of thoracic arms and said central portion.

15. The spinal decompression table according to claim 14, wherein said back support portion includes a central portion and a webbing portion on each side of said central portion relative to said longitudinal direction, said webbing portion on each side of said central portion coupling said central portion with an arm of said at least one pair of pelvic arms and providing flexibility of movement between said at least one pair of pelvic arms and said central portion.

16. The restraint system according to claim 13, wherein said at least one pair of pelvic arms is configured to extend around a pelvis of a patient such that said respective distal ends of said at least one pair of pelvic arms are substantially aligned and said at least one pair of thoracic arms is configured to extend around a thorax of a patient such that said respective distal ends of said at least one pair of thoracic arms are substantially aligned.

17. The restraint system according to claim 13, wherein said back support portion includes a first portion and a second portion arranged in said longitudinal direction, said thoracic portion being coupled with said first portion and said pelvic portion being coupled with said second portion.

18. A method of restraining a patient to a spinal decompression table, the method comprising the steps of:
positioning a patient on a back support portion which defines a longitudinal direction and is attached to a table frame;
positioning at least one pair of arms coupled with and extending from said back support portion around a body of said patient such that a pair of respective distal ends of said at least one pair of arms is substantially aligned relative to said longitudinal direction;
removably coupling said pair of distal ends of said at least one pair of arms with a securing mechanism; and
applying a predefined amount of tension through a biasing member to said securing mechanism to firmly secure said body of said patient to the spinal decompression table.
